(19) United States
(12) Patent Application Publication
(10) Pub. No.: US 2020/0077676 A1
(43) Pub. Date: Mar. 12, 2020

(54) METHODS FOR PRODUCING FEATHER-BASED FOOD PRODUCTS

(71) Applicant: MARS, INCORPORATED, McLean, VA (US)

(72) Inventors: Lucio Hiroshi Yonemoto, Franklin, TN (US); Svetlana Zivanovic, Franklin, TN (US); Ping Guo, Franklin, TN (US)

(73) Assignee: MARS, INCORPORATED, McLean, VA (US)

(21) Appl. No.: 16/613,186

(22) PCT Filed: May 29, 2018
(86) PCT No.: PCT/US2018/034951

§ 371 Date: Nov. 13, 2019

Related U.S. Application Data
(60) Provisional application No. 62/531,189, filed on Jul. 11, 2017.
(60) Provisional application No. 62/512,466, filed on May 30, 2017.

(51) Int. Cl. A23J 3/34; C07K 14/465; C07K 1/14; A23K 10/26; A23K 20/147; A23K 50/40; A23L 33/125; A23L 33/18; A23L 33/22; A23K 10/14; A23L 27/00

(52) U.S. Cl. CPC A23J 3/341; C07K 14/465; C07K 1/145; A23K 10/26; A23K 20/147; A23V 2002/00; A23L 33/125; A23L 33/18; A23L 33/22; A23K 10/14; A23L 27/84; A23K 50/40

(57) ABSTRACT

A method is provided for making food product ingredients from indigestible keratinous protein-containing material. The methods generally include adding an amount of cereal bran and/or one or more reducing sugars to the keratinous protein-containing material to provide a mixture and hydrolyzing the mixture. The methods generate fewer unpleasant odors, and food product ingredients produced by the method can similarly benefit. Antioxidants may also be added, and in such embodiments, even fewer off odors may be generated and/or palatability may be enhanced.

### FIELD

There are provided methods for processing keratin-containing proteinaceous materials such as feathers to produce digestible food and feed products. More particularly, the provided methods reduce or eliminate the odor associated with conventional hydrolysis methods and/or the odors or off flavors provided in food product ingredients produced therefrom.

### BACKGROUND

World population growth is well known to exert corresponding pressure on the food supply. As the population increases, already costly food ingredients, such as meat protein, may become prohibitively expensive for consumption by pets and companion animals. Thus, there is a need for alternative protein sources that do not compete with the human food chain. Such alternative protein sources may include any protein material containing keratin, including but not limited to feathers, hair, wool, hide, bristles, horns, hooves, claws, nails, scales, or any other suitable keratin-containing material or mixtures thereof.

While keratin protein materials are generally abundant, inexpensive, and sustainable, they also contain relatively high percentages of sulfur-containing amino acids such as cysteine. Cysteine can form disulfide bonds which contribute to the tertiary structure of the keratin protein, making it strong and durable. This structural durability also impairs digestibility, however, and in order to render keratin proteins digestible, at least partial breakdown of the disulfide bonds is necessitated.

Chemical, enzyme and heat hydrolysis has been used to denature keratin-containing materials and disrupt the sulfide bonds therein. Any of these can result in the formation of significant off odorants, including e.g., organosulfur compounds such as mercaptans and hydrogen sulfide. As a result, the environment in a facility wherein hydrolysis reactions are conducted can be suboptimal, and can even present a health hazard for employees that are sensitive to such odorants. Further, even if off gases from a hydrolysis process are scrubbed prior to or during venting, manufacturing facilities that perform hydrolysis reactions can have difficulty regulating the release or permeation of odors into the surrounding community. Some amount of off odorants or flavorants may also be present in food products incorporating hydrolyzed keratin materials. In food products that may already have some level of off odorants or flavorants, such as wet pet foods, these additional off odorants or flavorants can limit the consumer acceptance of those foods.

Known methods of ameliorating the odors associated with the hydrolysis of free peptides include the addition of reducing sugars in relatively large amounts, e.g., 20% or more, or ratios of peptides to reducing sugars of about 1.1. However, such additions can lead to other undesirable reactions, like sugar pyrolysis and caramelization, and any impact on the hydrolysis of intact proteins is not expected or understood. Further, the inclusion of such large amounts of such sugars may not be acceptable in all contemplated end uses of the hydrolyzed keratin material. This is particularly true since many conventional keratin hydrolysis processes lead to production of undesirable artificial amino acids, such as lanthionine, lysinoalanine, and other detrimental compounds well known in the art, in the end products. The addition of other undesirable ingredients is thus suboptimal.

Accordingly, there is a need for a process that will convert keratin-containing proteinaceous materials such as feathers into a desirable food product ingredient while reducing or eliminating the odor associated with the processes and food products incorporating the hydrolyzed keratin materials. Further benefit would be provided if tire processes employ nutritionally advantageous components.

### BRIEF DESCRIPTION

In one aspect, a process for producing a food protein ingredient from a keratinous material is provided. The process comprises adding an amount of cereal bran, a reducing sugar or a combination of these, to a quantity of keratinous protein-containing material to provide a mixture. The mixture is subjected to hydrolysis under conditions sufficient to hydrolyze the protein-containing material therein.

The cereal bran may be derived from any cereal source, including, but not necessarily limited to, amaranth, bulgur, farro, quinoa, spelt, teff, triticale, wild rice, wheat, corn, barley, rye, millet, oat, rice, sorghum, or buckwheat. In some embodiments, the cereal bran is selected from wheat, corn, barley, rye, millet, oat, or rice bran, and in certain advantageous embodiments, may be defatted rice bran.

Any reducing sugar capable of participating in Malliard reactions is suitable, including all monosaccharides (galactose, glucose, glyceraldehyde fructose, ribose and xylose), some disaccharides (cellobiose, lactose, and maltose), oligosaccharides (glucose syrup, maltodextrin and dextrin) and polysaccharides (glycogen). Of these, xylose has proven particularly advantageous and is used in some embodiments, alone or in combination with the cereal bran.

The cereal bran may be added to the keratinous protein-containing material in any suitable amount, in one or more additions. Desirably, the cumulative amount of cereal bran added during the method, in any number of additions, will be less than 20 wt %, or less than 10 wt. %. In some embodiments, the amount of cereal bran in the mixture will be from 0.1 wt. % to 20 wt. %, or from 5 wt. % to 10 wt. %. The weight percentages referenced herein are based upon the total dry weight of the keratinous protein-containing material and cereal bran.

Similarly, the reducing sugar may be added to the keratinous protein-containing material, or the mixture of keratinous protein-containing material and cereal bran, in any suitable amount, in one or more additions. Surprisingly low amounts of reducing sugars have proven to be effective, and amounts of less than 20 wt. %, or less titan 10 wt. %, or even less than 5 wt. % or even less than 1 wt. % are suitable. In some embodiments, the amount of reducing sugar, e.g., xylose, will be from 0.1 wt. % to 10 wt. %.

In those embodiments wherein a combination of cereal bran and reducing sugar(s) is used, the combined amount can also suitably be less than 20 wt. %, or less than 10 wt. %, or less than 5 wt. %. In some embodiments, the combined amount of cereal bran and reducing sugar can be 2 wt. % or less.

The keratinous protein-containing material may be any protein material containing keratin, including but not limited to feathers, hair, wool, hide, bristles, horns, hooves, claws, nails, scales, or any other suitable keratinous protein-containing material or mixtures thereof. The keratinous protein-containing material may further comprise one or more hydrolysates, or partial hydrolysates of any keratinous protein-containing material. In some embodiments, the keratinous protein-containing material comprises raw feathers.

The hydrolysis of the method may be conducted according to any known process, including steam, enzyme, chemical hydrolysis, or combinations thereof. Suitable conditions for steam hydrolysis include a pressure of from about 0 psig to about 200 psig and/or elevated temperatures, e.g., from 100 C. to 160 C., for a period of from about 15 minutes to about 240 minutes. Suitable conditions for enzyme hydrolysis include incubation with a suitable enzyme, or enzyme solution for a time and at a temperature sufficient to hydrolyze the keratinous protein-containing material/cereal bran mixture. Suitable enzymes include endoproteases, such as, e.g., keratinase, papain and combinations of these, exoproteases, endogenous enzymes, and combinations thereof.

One or more food grade antioxidants may be added to the keratinous protein-containing material, either before, during or after hydrolysis. The inclusion of such antioxidants may assist not only in the further reduction of oil odors associated with fat oxidation, but also, may surprisingly render the resulting food protein ingredient and thus a food product incorporating the same, more palatable. If desired, one or more food grade antioxidants may be included in the food protein ingredient in amounts suitable according to food and feed regulations, e.g., in amounts of from 0.01 wt. % to 10 wt. % based upon the total weight of the food protein ingredient.

The keratinous protein-containing material/cereal bran and/or reducing sugar mixture may be subjected to one or more pre-processing, intermediate or post processing steps conventionally used during hydrolysis processes. For example, the hydrolyzed mixture may be further processed by centrifugation, filtration, decanting, drying, sifting, accumulating prior to milling, concentrating, refrigerating, freezing, pasteurizing, acidifying, further hydrolyzing, and combinations thereof.

The inventive methods result in the minimized production of unpleasant odors during the manufacture of food product ingredients based upon keratinous protein-containing materials. Food products incorporating the food product ingredients produced by the method are expected to similarly benefit, i.e., and have minimal unpleasant odors associated therewith Further food products incorporating the food product ingredients may exhibit fewer or lesser degrees of any off flavors that may be exhibited by food products incorporating food product ingredients produced from keratinous protein-containing materials produced conventionally.

And so, in another aspect a food product ingredient is provided The food product ingredient can be one produced by the present methods, or stated another way, the food product ingredient can consist of a hydrolyzed mixture of keratinous protein-containing material and cereal bran and optionally, an amount of one or more antioxidants. The food product ingredient can include up to 20 wt. % cereal bran, and in some embodiments, desirably includes from 5 wt. % to 10 wt. % cereal bran, based upon the total weight of the food product ingredient. The food product ingredient, in turn, can be incorporated into a wet or dry food in amounts of up to 25 wt. %, or up to 20 wt. %, or up to 15 wt. %, or up to 10 wt. %, based upon the total weight of the food. At least 1 wt. % of the (bod product ingredient may be incorporated into a wet or dry food, or at least 5 wt. %. Acceptable ranges of the food product ingredient in wet or dry foods are from 1 wt. % to 25 wt. %, or from 5 wt. % to 20 wt. %, or from 10 wt. % to 15 wt.%. The weight percents provided herein are based upon the total weight of the food product ingredient intermediate mixture, food product ingredient, or food product, as the case may be, and are calculated on a dry matter basis.

### DESCRIPTION OF THE FIGURES

Various objects and advantages of this process and its compositions will become apparent from the following description taken in conjunction with the accompanying drawings which set forth, by way of illustration and example, certain embodiments of the process and resulting compositions.

FIG. 1 is a flow chart of one embodiment of the method;

FIG. 2 is a flow chart of an additional embodiment of the method;

FIG. 3 is a flow chart of an additional embodiment of the method; and

FIG. 4 is a flow chart of an additional embodiment of the method.

### DETAILED DESCRIPTION

The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced item.

The terms “about”, “approximately” and “substantially” are intended to signify that the item being qualified is not limited to the exact value specified, but includes for example, some slight variations or deviations therefrom, caused by measuring error, manufacturing tolerances, stress exerted on various parts, wear and tear, or combinations of these.

Reference throughout live specification to “one example” or “an example” means that a particular feature, structure, or characteristic described in connection with an embodiment or example is included in at least one embodiment. Thus, the appearance of the phrases “in one example” or “in an example” in various places throughout the specification does not necessarily indicate reference to the same embodiment. Further, the particular features, structures or characteristics may be combined in any suitable manner in one or more embodiments. And so, similarly, the phrase “m one embodiment” m various places m the specification are not necessarily referencing the same embodiment, although the inventive concepts disclosed herein are intended to encompass all combinations and permutations including one or more features.

Methods are provided for producing keratin-based protein ingredients for food and feed products and the resultant food protein ingredient. The methods employ a quantity of keratinous protein-containing material which may include any protein material containing keratin, including but not limited to feathers, hair, wool, hide, bristles, horns, hooves, claws, nails, scales, or any other suitable keratinous protein-containing material or mixtures thereof. Any of these may be obtained from a slaughterhouse or other source, and in the instance of hair and feathers, may be obtained from living animals that have shed the hair or molted the feathers.

Any of these may also be provided in a damp or wet condition, and as such, may desirably be dewatered by draining, sieving or the like to remove excess water. Dewatered keratin based protein ingredients may typically have a moisture content of from about 65% to about 80%.

The dewatered material is next transferred to a continuous or conveying bin where the material is aerated, agitated or otherwise decompacted, and conveyed to a contaminant separation station where organic and/or inorganic contaminants are separated from the protein-containing material to reduce or eliminate damage to subsequent processing equipment or contamination of the processed foodstuff product.

Cereal bran and/or one or more reducing sugars is/are added to the protein-containing material, and may desirably be added prior to any processing steps, upon receipt of the raw material, after any dewatering step, or before or after any contaminant removal step. The cereal bran and/or reducing sugar(s) is/are desirably added prior to any hydrolysis step, but may be added in portions before and after one or more hydrolysis steps of the method. The cereal bran and reducing sugar may be combined prior to addition to the protein-containing material, or may be added to the protein-containing material separately and/or sequentially.

The cereal bran may be de-fatted, if desired. Bran from any cereal may be used, such as amaranth, bulgur, farro, quinoa, spelt, teff, triticale, wild rice, wheat, com, barley, rye, millet, oat, rice, sorghum, or buckwheat bran. In some embodiments, the cereal bran may he wheat, corn, barley or rice bran. In some embodiments, the cereal bran is rice bran, which may or may not be defatted. The use of defatted cereal bran can provide further advantages in that the fat portion of the cereal bran can be separated and used in other products. Those embodiments in which defatted cereal bran is used can thus provide additional economic benefit.

The cumulative amount of cereal bran added during the method, in any number of additions, will desirably be no more than 20%, or no more than 18%, or no more than 16%, or no more than 14%, or no more than 12%, or no more than 10%, by weight of the total weight of the mixture of keratinous protein-containing material and cereal bran. The cumulative amount of cereal bran will desirably be at least 0.1%, or at least 0.5%, or at least 1%, or at least 2%, or at least 3% or at least 4% or at least, 5%, by weight of the total w eight on a dry matter basis, of the mixture of keratinous protein-containing material and cereal bran. In some instances, the amount of cereal bran in the mixture will be from 0.1 wt. % to 20 wt. %, or from 0.5 wt. % to 18 wt. %, or from 1 wt. % to 16 wt. %, or from 2 wt. % to 14 wt %, or from 3 wt. % to 12 wt. %, or from 5 wt. % to 10 wt. %.

Any reducing sugar capable of participating in Malliard reactions is suitable, including all monosaccharides (galactose, glucose, glyceraldehyde, fructose, ribose and xylose), some disaccharides (cellobiose, lactose, and maltose), oligosaccharides (glucose syrup, maltodextrin and dextrin) and polysaccharides (glycogen). Of these, xylose has proven particularly advantageous and is used in some embodiments, alone or in combination with the cereal bran.

Surprisingly low amounts of reducing sugars have proven to he effective, and amounts of less than 20 wt. %, or less than 10 wt. %, or even less than 5 wt. % or even less than 1 wt. % are suitable. In some embodiments, the amount of reducing sugar, e.g., xylose, will be from 0.1 wt. % to 1.0 wt. %

In those embodiments wherein a combination of cereal bran and reducing sugar(s) is used, the combined amount can also suitably be less than 20 wt. %. or less than 10 wt. %. or less than 5 wt. %. In some embodiments, the combined amount of cereal bran and reducing sugar can be 2 wt. % or less.

Pretreatment of the protein-containing material with proteolytic enzymes and suitable reducing agents may or may not be employed prior to hydrolysis. For example, a food grade reducing chemical such as sodium metabisulfite may be added to the keratinous protein-containing material, the mixture comprising the cereal bran and the keratinous protein-containing material to facilitate hydrolysis.

The mixture comprising the cereal bran and/or reducing sugar find keratinous protein-containing material is then subjected to hydrolysis under conditions sufficient to hydrolyze the protein-containing material therein, i.e., to break the disulfide bonds and denature the keratin protein. Any suitable type of hydrolysis may be performed, including steam, enzyme and/or chemical hydrolysis. Multiple hydrolysis steps may be performed, in such embodiments; each hydrolysis step may be performed using the same, or a different, hydrolysis method.

Multiple steam hydrolysis steps, multiple enzyme hydrolysis steps, multiple chemical hydrolysis steps, or combinations of these, may be used. Where more than one hydrolysis step is employed, the same, or different hydrolysis processes may be used. Variations of the same hydrolysis process may also be used. Variations in the processes may include variations in the conditions including retention times, pressures, temperatures, variations in the types of enzymes, or any combination of these. Multiple hydrolysis steps may be desirable to further enhance or maximize the digestibility of the final hydrolysate.

Suitable steam hydrolysis conditions comprise saturated steam at a pressure of from about 1 bar or 14.7 psig to about 4 bars or 58.8 psig and corresponding elevated temperatures, which may be determined based on known saturated steam properties. Heat may be supplied in indirect form through a high pressure vessel jacket, or it may be directly provided by steam heating. The protein-containing material/cereal bran and/or reducing sugar mixture is subjected to steam hydrolysis for a predetermined period of time to achieve a desired level of digestibility, generally from about 15 to about 60 minutes, or from about 15 to about 90 minutes, or from about 15 to about 126 minutes, or from about 15 minutes to about 150 minutes, or from about 15 minutes to about 180 minutes, or from about 15 minutes to about 210 minutes, or even from about 15 minutes to about 240 minutes.

The protein-containing material cereal bran and/or reducing sugar mixture may be agitated during steam hydrolysis, such as by shaking or stirring. Stirring may be employed to provide substantially continuous mixing, which facilitates penetration of the pressurized steam to achieve even heat throughout the mixture. Hydrolysis of the mixture may be accomplished using a continuous operation steam pressure hydrolyser system or a batch process type system. Once hydrolysis has been performed in accordance with predetermined pressure, temperature and time parameters, the mixture is discharged into an expansion tank where pressure and excess moisture are released. This generally brings the temperature of the mixture down to about 208-216° F. and preferably about 212° F. Preferably, the cooled mixture has a retained moisture content greater than about 40% to about 75%.

Enzyme hydrolysis can be carried out using any proteolytic enzyme known in the art, including but not limited to, proteases, such as endoproteases and exoproteases: exogenous enzymes: endogenous enzymes: or combinations thereof. Endoproteases, such as keratinase and papain, may be used either alone or in combination. Use of a combination of proteases may synergistically hydrolyze the keratin, providing efficiencies to the process.

Exoproteases may also be used in whole or as part of any enzyme hydrolysis to further reduce protein size, to generate peptides of desired characteristics, and/or to produce hypoallergenic and/or anallergenic protein ingredients. Any suitable enzyme products containing purified exoproteases may be employed, for example, Flavorzyme® (Novo Nordisk, Bagsvaerd, Denmark) and Validase FP® (DSM, Heerland, Netherlands). Alternatively, endogenous enzymes carried in raw material may be used to reduce the required dosages of added endoproteases. These may be obtained from animal viscera, for example, proteases, carbohydrases and/or lipases.

The enzymatic hydrolysis conditions are selected to produce optimum results, and are dependent on the enzymes employed. Agitation rate, moisture content, pH and temperature are selected in accordance with selected enzyme(s), and incubation conditions are tailored to achieve optimum results Higher hydrolysis temperatures may be employed where they increase the conversion rate without generating anti-nutrients such as lysinoalanine and lanthionine.

Duration of the enzymatic hydrolysis step is dependent on the starting material as well as the desired end product, but may last up to about 6 hours and preferably from about 30 minutes to about 6 hours. In order to maintain the commercial viability of the process, in certain preferred embodiments hydrolysis time is limited to less than about 4 hours. In certain particularly preferred embodiments, the hydrolysis time may range from about 2 hours to about 3 hours. In other embodiments, the hydrolysis time may be from about 30 minutes to about 2 hours.

One or more food grade antioxidants may he added to the keratinous protein-containing material, either before, during or after hydrolysis. In some embodiments, the antioxidant(s) may be added prior to or during hydrolysis. In other embodiments, the antioxidant(s) may be added after hydrolysis and prior to drying. The inclusion of such antioxidants may assist not only in the further reduction of off odors associated with fat oxidation, but also, has surprisingly been found to render the resulting food protein ingredient and thus a food product incorporating the same, more palatable.

Examples of food grade antioxidants that may be included in the food protein ingredient include any known food grade antioxidant, including, but not necessarily limited to, carotenoids such as beta-carotene, lutein, astaxanthin, zeaxanthin, bixin and lycopene; selenium; coenzyme Q10, lutein, tocotrienols; soy isoflavones; S-adenosylmethionine, glutathione; taurine; N-acetylcystein, vitamin E; vitamin C; vitamin A; lipoic acid; L-carnitine; propyl galate; ascorbyl palmitate; lecithin, tocopherol and mixed tocopherols; polyphenols such its oil of rosemary, rosemary extract, rosemarinic acid, cocoa polyphenols, or polyphenols found in tea or green tea coffee extract, coffeic acid, turmeric extract, blueberry extract, grapeseed extract; butylated hydroxyanisole (BHA), ternary butylhydroquinone (TBHQ), butylated hydroxytoluene (BHT), compounds containing one or more phenolic groups, carboxyl groups, lactone rings and/or isoprene units, or combinations of these. Examples of commercially available antioxidants acceptable for use in food include PET-OX® Premium Liquid (Kemin Industries, Des Moines, Iowa) and NATUROX® Liquid (Kemin Industries, Des Moines, Iowa).

If desired, one or more food grade antioxidants may be included the food protein ingredient in amounts according to food and feed regulations, e.g., in amounts of from 100 ppm to 10000 ppm on a dry matter basis, or from 0.01 wt. % to 1.0 wt % based upon total weight of the food protein ingredient.

After hydrolysis, the hydrolyzed material can he further processed according to conventional hydrolysis processes. This can include subjecting the material to one or more of drying, sifting, milling, comminuting, concentrating, refrigerating, freezing, pasteurizing, acidifying, centrifugation, filtration and or ultrafiltration, and or decanting. It is understood that this list is not exhaustive find further understood that not all further processing steps need to be performed in every embodiment of the method Alternatively, the hydrolyzed product may be directly dried.

For example, the hydrolyzed mixture may be transferred to a dryer feeder for supply to a dryer unit for moisture removal to render the product stable at ambient temperature and storage conditions. The dried food product ingredient may suitably have a moisture content of below about 10%, preferably of about 7.5% by weight. Any suitable type of dryer may be employed, such as a disc dryer or flash dryer. Dryer temperature and exposure time should be minimized to prevent darkening and decreased digestibility. Other drying technology known in the industry may also be used, either alone or in combination, including, but not limited to, spray drying or fluidized layer drying. One step gentle mill drying is one example of a particularly advantageous drying method.

The mixture may next be sifted and transferred to an accumulation or holding bin. If desired, the material may be subject to further milling or comminuting, or be subjected to additional contaminant removal techniques, such as magnetic metal separation via high intensity magnet bars or rods. The mixture is next transferred to cooling and dry bulk storage to await use.

As may be desirable for some end use applications, the keratinous protein-containing material, the mixture comprising the cereal bran and/or reducing sugar and the keratinous protein-containing material and/or the hydrolyzed mixture may be subjected to one or more size reduction steps, before, during or after any step of the process. Any such size reduction may be performed under wet conditions, dry conditions or any other conditions suitable to effect a size reduction. Any such size reduction may be completed in a single or multiple pass operation, which may include one, two, three, four, or any number of size reduction steps, to achieve a desired average particle size, or a desired D90, e.g., such as below about 400 μm.

The process of the present invention may result in the production of less off odorants than conventional processes for the production of food product ingredients from keratinous protein-containing material. This benefit may carry forward to products produced by the process, as well as downstream products incorporating the food product ingredients. That is, a food product ingredient produced by the method may outgas less off odorants than a food product ingredient produced from keratinous protein-containing materials by conventional process, and downstream products incorporating the food product ingredients produced by the method may similarly benefit. The food product ingredient may be incorporated into any end use food product, and particular advantage may be found in the incorporation of the food product ingredient into animal feeds, where high volume, and yet readily available, economical, nutritious, highly digestible and palatable protein sources are sought after. With less off odorants, a larger amount of keratinous material can be used than could be used without the present invention.

The food product ingredient may be particularly beneficial when incorporated into wet animal feeds, fish food, or pet foods that may emit off odorants as conventionally produced. That is, use of the food product ingredient in such products, wherein the aroma associated therewith may not be attractive to all users/consumers thereof may at least not add to any perceived malodor, as may be the case when food product ingredients containing keratinous protein-containing materials processed conventionally are incorporated into such products.

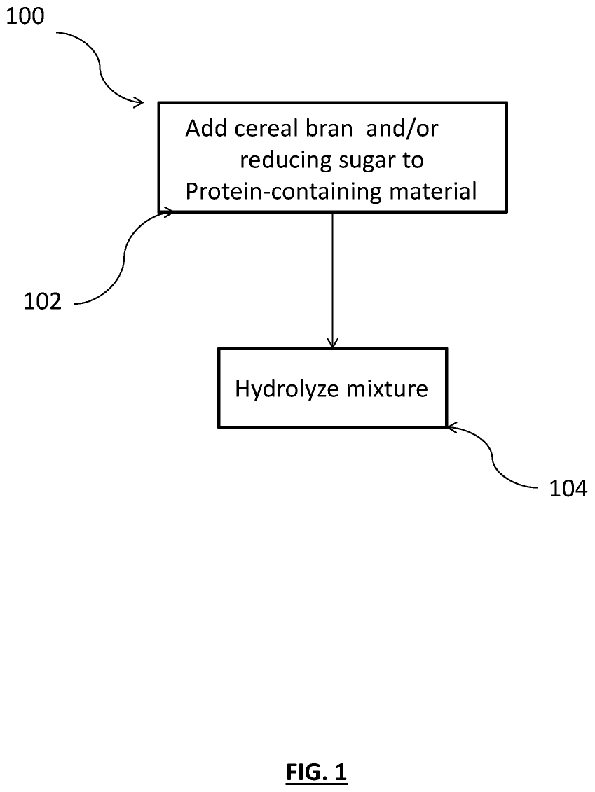

One embodiment of the method is shown in FIG. 1. As shown, method 100 generally involves adding 102 an amount of cereal bran, e.g., rice bran, and or a reducing sugar, e.g., xylose to a quantity of keratinous protein-containing material, e.g., feathers, to provide a mixture and hydrolyzing the mixture 104, e.g., using steam hydrolysis.

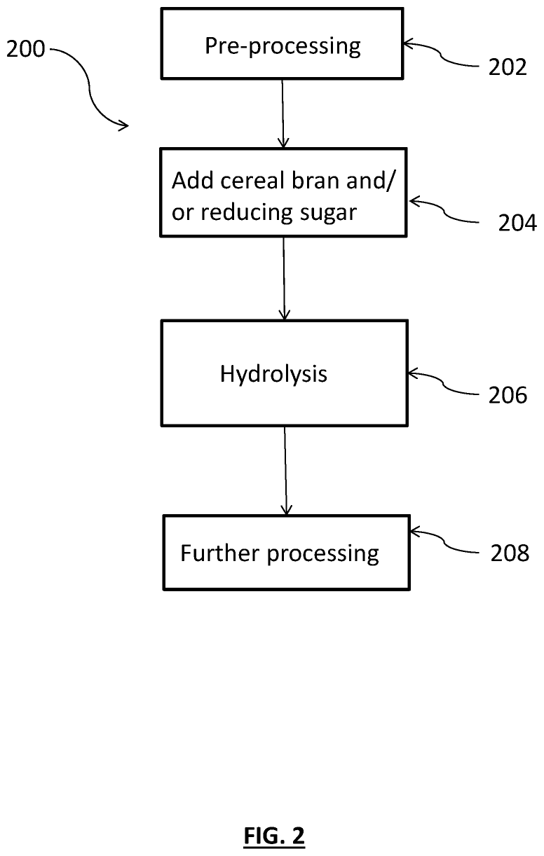

FIG. 2 illustrates a further embodiment of the method. In method 200, the keratinous protein containing material may be subjected to one or more pre-processing steps 202, such as removal of any organic or inorganic contaminants, wetting, de-watering, sieving, rinsing, size reduction, addition of proteolytic enzymes or reducing agents, etc. An amount of cereal bran, such as amaranth, bulgur, farro, quinoa, spelt, teff, triticale, wild rice, wheat, corn, barley, rye, millet, oat, rice, sorghum, or buckwheat bran and/or reducing sugar, such as galactose, glucose, glyceraldehyde, fructose, ribose, xylose, cellobiose, lactose, maltose, glucose syrup, maltodextrin, dextrin or glycogen is added 204 to the protein-containing material. Amounts of cereal bran from 0.1 wt. % to 20 wt. %, or from 1 wt. % to 15 wt. %, or from 5 wt. % to 10 wt. %, based upon the total weight of the mixture of protein containing material and cereal bran, are suitable. Similarly, amounts of reducing sugar(s) of less than 20 wt. %. or less than 10 wt. %, or even less than 5 wt. % or even less than 1 wt. % are suitable. In some embodiments, the amount of reducing sugar, e.g., xylose, will be from 0.1 wt. % to 1.0 wt. %. based upon the total weight of the mixture of protein-containing material and cereal bran and/or reducing sugar.

The protein containing material cereal bran and/or reducing sugar mixture is then hydrolyzed 206. This hydrolysis can be any hydrolysis process including, but not limited to steam hydrolysis, chemical hydrolysis, enzymatic hydrolysis, or combinations of these. The hydrolysis steps can use the same process, or they can use different processes (such as enzymatic or steam) or different processing conditions (e.g. different enzymes, different pressures, different temperatures, different retention times, etc.).

The hydrolyzed feathers are then subjected to further processing 208, which may involve any step that is performed in a hydrolysis process. For example, the hydrolyzed feathers can be subjected to one or more size reduction processes, dried, sifted, and accumulated prior to milling and being placed in dry bulk storage, or they may be concentrated, refrigerated, frozen, pasteurized, acidified and/or subjected to further hydrolysis.

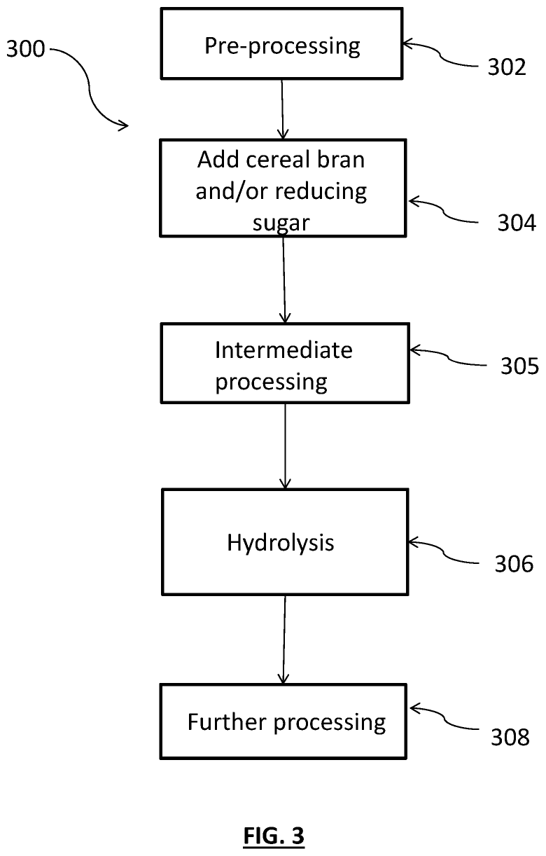

FIG. 3 shows yet another embodiment of the method 300, wherein one or more intermediate processing steps 305 are conducted between the addition of cereal bran and/or reducing sugar 304 and hydrolysis 306. Intermediate processing steps 305 may include removal of any organic or inorganic contaminants, wetting, rinsing, size reduction, addition of proteolytic enzymes or reducing agents, etc.

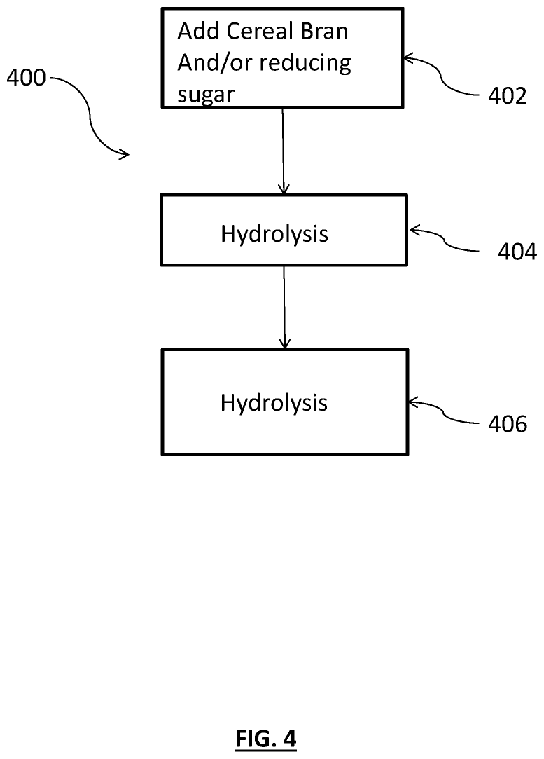

As shown in FIG. 4, another alternate method 400 involves the steps of adding 402 cereal bran and/or reducing sugar, hydrolysis 404, and a second hydrolysis 406. The hydrolysis processes 404, 406 may use the same process, or one or more of the hydrolysis steps may use a different hydrolysis process or a variation of the same hydrolysis process. Variations in the processes may include variations in the conditions including retention times, pressures, temperatures, variations in the types of enzymes, or any combination of these variations.

The hydrolyzed mixture can then again be subjected to a contaminant removal step, wherein any foreign materials are separated using X-ray or other suitable sorting means. Removal of such inclusions serves to prevent damage to cutting head equipment as well as contamination of the feedstock product.

The food product ingredient made in accordance with the foregoing methods is stable at room temperature, highly palatable and exhibits at least about 85%, or at least about 87%, or at least about 89%, or at least about 91%, or at least about 92%, or at least about 93%, or at least about 94%. or at least about 95%, or at least about 96%, or at least about 97%, or at least about 98%, or at least about 99%, or even at least about 99.5% protein digestibility as measured by the 2-step enzymatic method described by Boisen and Fernandez (1995). In some embodiments, the food product ingredient exhibits between 85%-95% protein digestibility as measured by the 2-step Boisen method.

The food product ingredient contains a variety of amino acids, including but not limited to Cysteine, Leucine, Arginine, Glutamic acid, Glycine, Serine and Phenylalanine.

The following examples describe embodiments wherein the protein-containing material comprised raw feathers and or raw feather hydrolysate. However, the methods are not so limited and can be applied to any keratinous protein-containing material, without limitation.

### EXAMPLE 1

### Pre-Treatment (Optional)

1818.2 g of sodium metabisulfite are dissolved in 10 kg of water to make a solution to he sprayed onto the surface of approximately 2000 pounds of feathers. The solution is sprayed onto layers of feathers as they are placed into a plastic container for transport. The packaged feathers are subjected to steam treatment for approximately 15 minutes to achieve a minimum temperature of 180° F. After this treatment, the feathers are transported and stored appropriately prior to the steam hydrolysis step. Optionally, the desired cereal bran and/or reducing sugar, in the desired amount could be added to the pretreatment solution or directly to the feathers prior to pre-treatment and packaging.

### Steam Hydrolysis

120 kg of the raw or pre-treated feathers alone (comparative), or with 12 kg added defatted rice bran (inventive) are placed into a pilot batch steam hydrolyzer. The batch hydrolyzer has a dual plate mounted on a single shaft to mix the feathers during hydrolysis. The shaft speed is kept at 18 rpm. The steam coming from a boiler is circulated through a jacket of the batch hydrolyzer. The feathers and feather rice bran mixture are heated in the vessel by the heat conducted from the jacket wail to the feathers or feather, rice bran mixture The steam pressure in the jacket is maintained in the range of 55 psig to 70 psig. As heat is transferred to mixture, the pressure in the vessel will build over time, but is manually adjusted to be maintained at 40 psig. The hydrolysis was carried out at 40 psig for 60 minutes. After the pre-determined time, the pressure of the vessel is released slowly by opening a manual valve until the pressure reached atmospheric. Next, the batch hydrolyzer is opened and hydrolyzed feathers and mixture are transferred to separate containers.

### Off-Gassing During Hydrolysis

Hydrolysis of the feathers alone (comparative) is expected to produce odors with typical sulfur-containing smells similar to a natural gas, or rotten egg, smell, while hydrolysis of the inventive sample, including defatted rice bran, is expected to produce a more delicate and less sulfury smell similar to a baked cereal odor.

### Measurement of Digestibility

Finally, the material from both containers was subjected to the 2-step Boisen method to determine the digestibility. As can be appreciated from the foregoing description, the only difference in processing between the two containers was the addition of defatted rice bran to the feathers for the inventive example. The addition of defatted rice bran did not detrimentally impact the digestibility of the food product ingredient, and may provide slight increases in digestibility.

### Drying

The dough-like material was dried to achieve shelf stable conditions. In this experiment, a hot air dryer provided by Scott Equipment, New Prague, Minn. was used. The dryer had a chamber heated by hot air passing through at high speed. The material was dispersed by a series of paddles placed on a single rotating shaft. The paddles also conveyed the material inside the drying chamber find could be adjusted as required. The hot air inlet temperature was set at 600° F. and the temperature was at 245° F. at outlet of the drying chamber. The dried material came out in fine powder format and was collected by a series of filters assembled in a “bag house”. The collected powder was conveyed through an air lock rotary valve and discharged into a container. The product temperature measured at this point was 135° F. The moisture was below 10%, with water activity low enough to be stored in ambient temperature. Those skilled in the art will appreciate that other drying technology could also be employed in association with the described method to produce high quality feather hydrolysate powder.

### Generation of Off Flavors/Odors

After storage for 24 hours, 48 hours, 72 hours, 1 week and 2 weeks, the comparative (hydrolysis of feathers alone) and inventive (hydrolysis of feathers with cereal bran) food product ingredients are incorporated into comparative and inventive pet food products, either in the same facility, or after transport transfer to a different facility/entity. The comparative pet food products are expected to have an aroma less acceptable to human consumer than the aroma of the inventive pet food products.

### Results

Food product ingredients produced according to the present method are expected not only to release less off odors during the manufacture thereof as compared to food product ingredients produced according to conventional methods, i.e., without the addition of cereal bran. Further, pet foods into which the food product ingredients are incorporated fire expected to experience greater consumer acceptance than food product ingredients prepared from keratinous materials using conventional methods. Further, pets are expected to prefer foods incorporating the inventive pet food ingredient as compared to pet foods including keratinous materials processed conventionally. Even further, the food products can include a greater amount of the inventive keratinous material than conventional keratinous materials.

### EXAMPLE 2

### Laboratory Scale Screening

One or more sugars or cereal bran, 200 g of feathermeal hydrolysates and 200 g water were mixed and reacted in a Parr reactor (Parr Instrument Co., Moline Ill.) at 120° C. for 30 minutes. Sensors evaluation was conducted on the processed wet samples by a panel of 3 human evaluators familiar with the off-odors generated by conventional feather hydrolysis, but not using any particular standardized sensory methodology. Samples were ranked on a scale of 1 to 5 where 5 is the best smelling (defined as mild and/or pleasant) find 1 is the worst smelling (defined as strong and/or unpleasant smelling). Sugar amounts, cereal bran amounts and sensory ratings are shown below in Table 1.

TABLE 1

| Sample | Feather | | Cereal | Sensory |
|---|---|---|---|---|
| ID | Type | Sugar wt. % | Bran, wt. % | Rating |
| 8/2-1 | Hydrolysate | — | — | 1.2 |
| 9/2-2 | Hydrolysate | Xylose, 0.5 wt. % | — | 2.3 |
| 12/2-3 | Hydrolysate | HFCS¹, 0.5 wt. % | — | 1.7 |
| 13/2-4 | Hydrolysate | HFCS + Xylose, | — | 1.0 |
| | | 0.5 wt. % each | | |
| 14/2-5 | Hydrolysate | Brewer's yeast, | — | 1.0 |
| | | 0.5 wt. % | | |
| 23/2-6 | Hydrolysate | Xylose, 1 wt. % | — | 2.7 |
| 10/2-7 | Hydrolysate | — | Defatted rice | 2.3 |
| | | | bran, 5 wt. % | |

¹High Fructose Corn Syrup

As shown in Table 1, the samples containing 0.5 wt. % xylose. 1 wt. % xylose and 5 wt. % defatted rice bran (samples 2-2, 2-6 and 2-7) scored the highest Comparative samples 2-1, 2-4 and 2-5, containing either no amount of sugar, a combination of HCFS and xylose or brewer's yeast, scored the lowest. It was thus decided to conduct pilot plant scale screening of defatted rice bran and xylose, with a comparative example of high fructose corn syrup. This screening is described in Example 3.

### EXAMPLE 3

### Pilot Plant Scale

Sugar or cereal bran was mixed with raw feathers in the amounts shown in Table 2. Each sample was processed in a Littleford Day Pilot Hydrolyzer at 140° C., 40 PSI for 15 minutes. Hydrolyzed feathers were comminuted using a Comitrol® model 1700 processor (Urschel Laboratories, Inc.) and dried using a Ring dryer at a drying temperature of 82° C. Sensory evaluation was conducted on the dried samples by a panel of 3 human evaluators, familiar with the off-odors generated by conventional feather hydrolysis, but without using any particular standardized sensory methodology. Samples were ranked on a scale of 1 to 5 where 5 is the best smelling (defined as mild and/or pleasant) and 1 is the worst smelling (defined as strong and/or unpleasant smelling). The resulting sensory ratings are shown below in Table 2.

TABLE 2

| | | | Raw | Initial | Final | |
|---|---|---|---|---|---|---|
| Sam- | | Additive | feather | Mois- | Mois- | |
| ple | | amount | amount | ture | ture | Sensory |
| ID | Additive | (kg) | (kg) | (%) | (%) | Rating |
| 3-1 | D-xylose | 0.15 | 15 | 64.8 | 69.2 | 2.5 |
| 3-2 | HFCS | 0.15 | 15 | 71.6 | 70.4 | 2.0 |
| | (comparative) | | | | | |
| 3-3 | Defatted | 1.7 | 15 | 75.6 | 60.1 | 3.0 |
| | Rice Bran | | | | | |

As shown in Table 2, the samples made using xylose or defatted rice bran scored better than the comparative example made with HFCS. More specifically, the panelists described the smell of sample 3-1 as a cooked sugar smell, the smell of sample 3-3 as a baked cereal smell, and the smell of comparative sample 3-2 being the same as hydrolyzed feathers produced conventionally. Furthermore, inventive sample 3-3 was described by the panelists as having an appealing light yellow, light brown color. Based upon these results, it was decided to conduct confirmatory sensory testing, using Flash Profiling methodology. This confirmatory testing is described in Example 4.

### EXAMPLE 4

### Confirmatory Testing

Sugar or cereal bran w as mixed with raw feathers in the amounts shown in Table 3. Each sample was processed in a Littleford Day Pilot Hydrolyzer at 140° C., 40 PSI for 15 minutes. Hydrolyzed feathers were comminuted using a Comitrol® model 1700 processor (Urschel Laboratories, Inc.) and dried using a Ring dryer at a drying temperature of 82° C. Sensory evaluation was conducted on the dried samples using Flash Profiling according to the methodology described at www.sensorysociety.org/knowledge/sspwiki/pages/FlashProfile.aspx.

Samples were ranked on a scale of 1 to 5 where 5 is the best smelling (defined as mild and or pleasant) and 1 is the worst smelling (defined as strong and/or unpleasant smelling), lire resulting sensory ratings are shown below in Table 3.

TABLE 3

| Sam- | | Inclusion | | | |
|---|---|---|---|---|---|
| ple | | level | | Inten- | Accept- |
| ID | Additive | (%) | Season Description | sity | ability |
| 4-0 | Control | — | Burnt, slight sulfur, | 3 | 3 |
| | | | meaty, barnyard | | |
| 4-1 | D-xylose | 0.5 | Sweet, roasted, nutty, | 2.3 | 4 |
| | | | meaty | | |
| 4-2 | D-xylose | 1.0 | Sweet, roasted, nutty, | 3 | 4.3 |
| | | | meaty | | |
| 4-3 | Glucose | 0.5 | Grain, slightly sweet, | 2.0 | 3.3 |
| | | | slight sulfur, slight | | |
| | | | meaty | | |
| 4-4 | Glucose | 1.0 | Barnyard, roasted, | 1.3 | 3.3 |
| | | | oily, burnt | | |
| 4-5 | Defatted | 1.0 | Barnyard, grain, | 2.3 | 3.3 |
| | Rice Bran | | slight sulfer | | |
| 4-6 | Defatted | 5.0 | Meaty, oily, roasted, | 2.7 | 3.3 |
| | Rice Bran | | sweet, grain | | |
| 4-7 | Defatted | 10.0 | Grain, sweet, roasted, | 2.3 | 4.3 |
| | Rice Bran | | meaty, hominy | | |
| 4-8 | Rice Bran | 1.0 | Grain, roasted, sweet, | 2.7 | 3.3 |
| | Fiber (RBF) | | meaty | | |
| 4-9 | Rice Bran | 5.0 | Sweet, meaty, roasted, | 2.3 | 4.0 |
| | Fiber (RBF) | | grain | | |
| 4-10 | Rice Bran | 10.0 | Sweet, grain, cooked | 3 | 4.3 |
| | Fiber (RBF) | | rice | | |

It is believed that the difference in scores between Examples 2, 3 and 4, is due to the different methodology employed for Example 4. Nonetheless, the three examples taken together clearly show that low amounts, e.g., 10 wt. % or less, had a positive impact on the odor associated with hydrolyzed protein-containing material.

### EXAMPLE 5

### Pre-Treatment (Optional)

1818.2 g of sodium metabisulfite are dissolved in 10 kg of water to make a solution to he sprayed onto the surface of approximately 2000 pounds of feathers. The solution is sprayed onto layers of feathers as they are placed into a plastic container for transport. The packaged feathers are subjected to steam treatment for approximately 15 minutes to achieve a minimum temperature of 180° F. After this treatment, the feathers are transported and stored appropriately prior to the steam hydrolysis step. The desired cereal bran and/or reducing sugar, in the desired amount, is added to the pretreatment solution or directly to the feathers prior to pre-treatment and packaging.

### Steam Hydrolysis

120 kg of the raw or pre-treated feathers alone (comparative), or with 12 kg (10 wt.%) added defatted rice bran (inventive), 1.2 kg (1 wt.%) xylose (inventive), and 10 kg defatted rice bran and 2 kg xylose (for a combined 10 wt.% of the mixture, inventive) are placed into a pilot batch steam hydrolyzer. The batch hydrolyzer has a dual plate mounted on a single shaft to mix the feathers during hydrolysis. The shaft speed is kept at 18 rpm. The steam coming from a boiler is circulated through a jacket of the hatch hydrolyzer The feathers and feather/rice bran, feather/xylose, and feather rice bran and xylose mixtures are healed in the vessel by the heat conducted from the jacket wall to the feathers or feather/rice bran feather/xylose, and feather/rice bran and xylose mixtures. The steam pressure in the jacket is maintained in the range of 55 psig to 70 psig. As heat is transferred to mixture, the pressure in the vessel will build over time, but is manually adjusted to be maintained at 40 psig. The hydrolysis was carried out at 40 psig for 60 minutes. After the pre-determined time, the pressure of the vessel is released slowly by opening a manual valve until the pressure reached atmospheric. Next, the batch hydrolyzer is opened and hydrolyzed feathers and mixtures are transferred to separate containers.

### Off-Gassing During Hydrolysis

Hydrolysis of the feathers alone (comparative) is expected to produce odors with typical sulfur-containing smells similar to a natural gas, or rotten egg, smell, while hydrolysis of the inventive samples are expected to produce a more delicate and less sulfury smell similar to a baked cereal odor.

### Measurement of Digestibility

Finally, the material from each container was subjected to the 2-step Boisen method to determine the digestibility. As can be appreciated from the foregoing description, the only difference in processing between the containers was the addition of defatted rice bran, xylose and defatted rice bran plus xylose to the feathers for the inventive examples. The addition of defatted rice bran, xylose, or defatted rice bran plus xylose did not detrimentally impact the digestibility of the food product ingredient, and may provide slight increases in digestibility.

### Drying

The dough-like material was dried to achieve shelf stable conditions. In this experiment, a hot air dryer provided by Scott Equipment, New Prague, Minn. was used. The dryer had a chamber heated by hot air passing through at high speed. The material was dispersed by a series of paddles placed on a single rotating shaft. The paddles also conveyed the material inside the drying chamber and could be adjusted as required. The hot air inlet temperature was set at 600° F. and the temperature was at 245° F. at outlet of the drying chamber. The dried material came out in fine powder format and was collected by a series of filters assembled in a “bag house”. The collected powder was conveyed through an air lock rotary valve and discharged into a container. The product temperature measured at this point was 135° F. The moisture was below 10%, with water activity low enough to be stored in ambient temperature. Those skilled in the art will appreciate that other drying technology could also be employed in association with the described method to produce high quality feather hydrolysate powder.

### Generation of Off Flavors/Odors

After storage for 24 hours, 48 hours, 72 hours, 1 week and 2 weeks, the comparative (hydrolysis of feathers alone) and inventive (hydrolysis of feathers with cereal bran, xylose and cereal bran and xylose) food product ingredients are incorporated into comparative and inventive pet food products, either in the same facility, or after transport/transfer to a different facility/entity. The comparative pet food products are expected to have an aroma less acceptable to human consumers than the aroma of the inventive pet food products.

### Results

Food product ingredients produced according to the present method are expected not only to release less off odors during the manufacture thereof as compared to food product ingredients produced according to conventional methods, i.e., without the addition of cereal bran, xylose and a combination of cereal bran and xylose. Further, pet foods into which the food product ingredients are incorporated are expected to experience greater consumer acceptance than food product ingredients prepared from keratinous materials using conventional methods. Further, pets are expected to prefer foods incorporating the inventive pet food ingredient as compared to pet foods including keratinous materials processed conventionally. Even further, the food products can include a greater amount of the inventive keratinous material than conventional keratinous materials.

It is to be understood that while certain forms of the method for producing food protein ingredient from keratinous protein-containing materials have been illustrated and described herein, it is not to be limited to the specific forms or arrangement of parts described and shown. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

1. A process for producing a food protein ingredient from a keratinous material, the process comprising:
adding an amount of a cereal bran and/or one or more reducing sugars to a quantity of a keratinous protein-containing material to provide a mixture; and
subjecting the mixture to hydrolysis under conditions sufficient to hydrolyze the keratinous protein-containing material therein.

2. The process of claim 1, wherein the cereal bran is amaranth, bulgur, farro, quinoa, spelt, teff, triticale, wild rice, wheat, corn, barley, rye, millet, oat, rice, sorghum, or buckwheat bran.

3. (canceled)

4. The process of claim 1, wherein the cereal bran is defatted.

5. The process of claim 1, wherein the cereal bran is added in an amount of 10 wt. % or less, based upon the total weight of the mixture.

6. (canceled)

7. The process of claim 1, wherein both the cereal bran and the one or more reducing sugars are added to the keratinous protein-containing material.

8. The process of claim 1, wherein the one or more reducing sugars comprises galactose, glucose, glyceraldehyde, fructose, ribose, xylose, cellobiose, lactose, maltose, glucose syrup, maltodextrin, dextrin or glycogen.

9. The process of claim 8, wherein the one or more reducing sugars is xylose.

10. The process of claim 1, wherein the one or more reducing sugars is added in an amount of 5 wt. % or less, based upon the total weight of the mixture.

11. (canceled)

12. The process of claim 1, wherein the keratinous protein-containing material comprises raw feathers.

13. The process of claim 1, wherein the keratinous protein-containing material is subjected to steam hydrolysis at a pressure of from about 0 psig to about 200 psig and/or elevated temperature for a period of from about 15 minutes to about 240 minutes.

14. The process of claim 1, wherein the keratinous protein-containing material is subjected to enzyme hydrolysis.

15. The process of claim 14, wherein the enzyme hydrolysis comprises:
adding a proteolytic enzyme slurry comprising a quantity of at least one proteolytic enzyme in an aqueous environment to the mixture to produce a protein slurry;
incubating the protein slurry for a time sufficient to produce the food protein ingredient.

16. The process of claim 15, wherein the proteolytic enzyme is selected from the group consisting of endoproteases, exoproteases, endogenous enzymes, and combinations thereof.

17. The process of claim 16, wherein the endoproteases include enzymes selected from the group consisting of keratinase, papain, and combinations thereof.

18. The process of claim 1, wherein the hydrolyzed keratinous protein-containing material is further processed by centrifugation, filtration, decanting, drying, sifting, accumulating prior to milling, concentrating, refrigerating, freezing, pasteurizing, acidifying, further hydrolyzing, or combinations thereof.

19. The process of claim 1, further comprising adding an amount of one or more antioxidants to the keratinous protein-containing material before, during or after hydrolysis.

20. A pet food comprising a food protein ingredient produced by the process of claim 1.

21. A food product ingredient consisting of a hydrolyzed keratinous material, a cereal bran and/or one or more reducing sugars, and optionally one or more antioxidants.

22. The food product ingredient of claim 21, wherein the cereal bran is present in an amount of 20 wt. % or less and/or wherein the one or more reducing sugars is present in an amount of 5 wt. % or less, based upon the total weight of the food product ingredient.

23. (canceled)

24. A method of removing malodors from and/or increasing the palatability of a keratinous material comprising the steps of adding an amount of cereal bran and/or one or more reducing sugars to a quantity of a keratinous protein-containing material to provide a mixture; subjecting the mixture to hydrolysis under conditions sufficient to hydrolyze the protein-containing material therein, and optionally adding an amount of one or more antioxidants to the keratinous protein-containing material before, during or after hydrolysis.

25. A pet food comprising the food product ingredient of claim 21, wherein the amount of the hydrolyzed keratinous material is higher than in a pet food product without cereal bran.

26. The pet food of claim 25, wherein the pet food is a wet pet food or a dry pet food, and wherein the food product ingredient is included in an amount of from 1 wt. % to 25 wt. %.

27. (canceled)
